(19) United States
(12) Patent Application Publication
(10) Pub. No.: US 2024/0290467 A1
(43) Pub. Date: Aug. 29, 2024

(54) METHOD AND SYSTEM FOR TRANSMITTING MEDICAL IMAGING DATA

(71) Applicant: L&T TECHNOLOGY SERVICES LIMITED, Chennai (IN)

(72) Inventor: HAJA NAZURUDEEN MOHAMED SHEIK KATHI, Thanjavur District (IN)

(21) Appl. No.: 18/209,528

(22) Filed: Jun. 14, 2023

(30) Foreign Application Priority Data
Feb. 23, 2023 (IN) 202341012388

(51) Int. Cl. G16H 30/20; G16H 10/60; G16H 30/40

(52) U.S. Cl. CPC G16H 30/20; G16H 10/60; G16H 30/40

(57) ABSTRACT

The invention relates a method and a system for transmitting medical imaging data from a source system to a receiver system. The method includes receiving encoded medical imaging data from the source system; decoding the encoded medical imaging data using a decryption technique; validating the decoded medical imaging data based on the plurality of predefined standards; upon successful validation, mapping the decoded medical imaging data based on one or more business objects associated with the receiver system; and storing the decoded medical imaging data in a data store associated with the receiver system based on one or more configurations of the receiver system.

### TECHNICAL FIELD

This disclosure relates generally to medical data exchanging systems, and more particularly to a system and method for transmitting medical imaging data from a source system to a receiver system.

### BACKGROUND

Today, exchange of DICOM images (Digital Imaging and Communications in Medicine) is possible between different medical systems. DICOM is a standard for storing and transmitting medical images, such as X-rays, Computed Tomography (CT) scans, and Magnetic Resonance Imaging (MRI) scans. The DICOM images include not only pixel data, but also additional information (for example, a patient name, a date of examination, and other relevant imaging parameters). This information allows for accurate interpretation of an image, and capability of transferring images between different medical systems or facilities. However, since the DICOM is a broad standard, it has certain limitations when it comes to image reconstruction with new parameters. Further, for image reconstruction, medical imaging data can be transferred from one medical system to another medical system. However, transferring the medical imaging data from one medical system to another medical system is not secure.

Further, for example, when a patient switches from one healthcare provider to another healthcare provider, the patient may be insisted to undergo another CT/MRI scan when the required orientations are not available in an existing DICOM image generated through a scanner associated with a previous healthcare provider. When the scan is completed again with contrast material, then there are chances of affecting Kidney health of the patient.

Therefore, there is a need for a platform for CT and MRI concept through which the medical imaging data can be transferred securely from a source system to receiver system. Further, the transferred data can be used by the receiver system to reconstruct medical images with required new parameters. Thus, use of the contrast material again with X-ray exposure can be avoided as examination can be performed at one place and reconstruction of the medical images with required parameters can be accomplished at any other place.

### SUMMARY OF THE INVENTION

In an embodiment, a method for transmitting medical imaging data from a source system to a receiver system is disclosed. The method may include receiving encoded medical imaging data from the source system. The encoded medical imaging data may include the medical imaging data in a machine-readable format and validated based on a plurality of predefined standards. The method may further include decoding the encoded medical imaging data using a decryption technique. The method may further include validating the decoded medical imaging data based on the plurality of predefined standards. The method may further include mapping the decoded medical imaging data based on one or more business objects associated with the receiver system, upon successful validation. The method may further include storing the decoded medical imaging data in a data store associated with the receiver system based on one or more configurations of the receiver system.

In another embodiment a system for transmitting medical imaging data from a source system to a receiver system is disclosed. The system may include a processor and a memory communicatively coupled to the processor. The memory may store processor-executable instructions, which, on execution, may cause the processor to receive encoded medical imaging data from the source system. The encoded medical imaging data may include the medical imaging data in a machine-readable format and validated based on a plurality of predefined standards. The processor-executable instructions, on execution, may further cause the processor to decode the encoded medical imaging data using a decryption technique. The processor-executable instructions, on execution, may further cause the processor to validate the decoded medical imaging data based on the plurality of predefined standards. The processor-executable instructions, on execution, may further cause the processor to map the decoded medical imaging data based on one or more business objects associated with the receiver system, upon successful validation. The processor-executable instructions, on execution, may further cause the processor to store the decoded medical imaging data in a data store associated with the receiver system based on one or more configurations of the receiver system.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 illustrates a block diagram of various modules within a source system, in accordance with an embodiment of the present disclosure.

FIG. 2 illustrates a block diagram of various modules within a receiver system, in accordance with an embodiment of the present disclosure.

FIG. 3 illustrates a block diagram of various modules within an exemplary image reconstruction system, in accordance with an embodiment of the present disclosure.

FIG. 4 illustrates a flow chart of a method for transmitting medical imaging data from a source system to a receiver system, in accordance with an embodiment of the present disclosure.

FIG. 5 illustrates a flow chart of a detailed method for transmitting medical imaging data from a source system, in accordance with an embodiment of the present disclosure.

FIG. 6 illustrates a flow chart of a method of reconstructing a medical image, in accordance with an embodiment of the present disclosure.

FIG. 7 illustrates a control logic for transmitting medical imaging data from a source system to a receiver system, in accordance with an embodiment of the present disclosure.

FIG. 8 is a block diagram of an exemplary computer system for implementing embodiments consistent with the present disclosure.

### DETAILED DESCRIPTION OF THE DRAWINGS

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims. Additional illustrative embodiments are listed below.

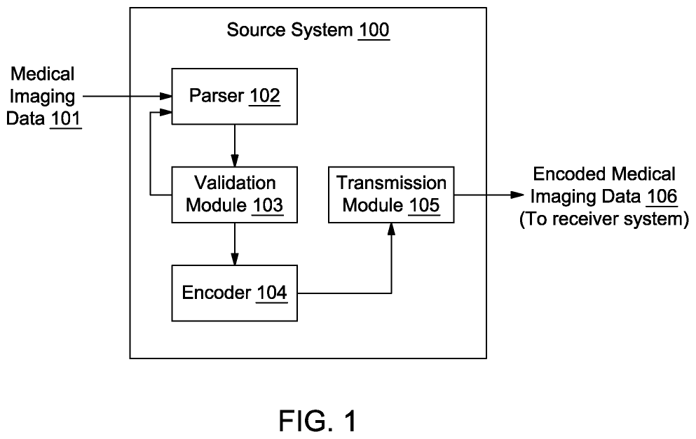

Referring now to FIG. 1, various modules within a source system 100 are illustrated, in accordance with an embodiment. The source system 100 generates encoded medical imaging data 106 which may be further transferred to a receiver system 200. To generate the encoded medical imaging data 106, the source system 100 includes a parser 102, a validation module 103, an encoder 104, and a transmission module 105. Further, the source system 100 may also include a data store (not shown in FIG. 1) to store intermediate results generated by the modules 102-105.

In some embodiments, the parser 102 may be configured to receive medical imaging data 101. In some other embodiments, the parser 102 may be configured to parse the medical imaging data 101 to generate the medical imaging data in the machine-readable format. Examples of the machine-readable format include, but are not limited to, Comma-Separated Values (CSV) format, JavaScript Object Notation (JSON) format, Extensible Markup Language (XML) format, and the like. It should be noted that the medical imaging data 101 includes at least one text data and binary data. Also, it should be noted that the medical imaging data 101 includes a set of variable fields and a set of constant fields.

For example, the set of constant fields may include at least one of text data and binary data corresponding to a patient, an imaging system, an institution where the source system is installed, cardiac data, pulmonary data, injector data, a prescribed medicine, and a certified technician. Further, for example, the set of variable fields comprises at least one of text data and binary data corresponding to an examination parameter, a scan preference, an image acquisition parameter, a bolus tracking detail, collimation data, a scan geometry, scan coordinates, one or more examination rules and constraints, and additional examination data. The parser 102 may be communicatively coupled to the validation module 103 to transmit the parsed medical imaging data.

The validation module 103 may be configured to receive the medical imaging data from the parser 102. Further, the validation module 103 may be configured for validating the medical imaging data based on the plurality of predefined standards. For example, the medical imaging data should include text data and binary data, and the format of the medical imaging data should be the machine-readable format. In other words, validation may be performed to check that the medical imaging data are converted as per the plurality of predefined standards. The plurality of predefined standards is common standards or mutually agreed standards of data transmission for the source system 100 and the receiver system 200. When the validation module 103 identifies that the medical imaging data is not compliant with the plurality of predefined standards, then the validation module 103 may indicate an error to a user/administrator with a reason for failure. This condition may be considered as a failed validation condition. In some embodiments, in case of the failed validation condition, the validation module 102 may send a signal to the parser 102 so that the modifications in the medical imagining data may be done or the medical imaging data may be parsed again.

When the validation module 103 identifies that the medical imaging data is compliant with the plurality of predefined standards, the validation module 103 may transmit the medical imaging data to the communicatively connected encoder 104.

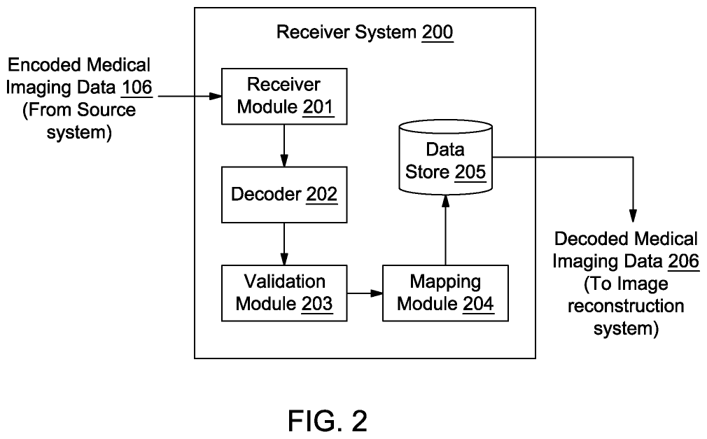

The encoder 104 may be configured for encoding the medical imaging data upon successful validation, thereby ensuring security the medical imaging data. The encoder 104 may use an encryption technique to generate the encoded medical imaging data 106. For example, the encryption technique may include, but is not limited to, an Advanced Encryption Standard (AES) technique, a Data Encryption Standard (DES) technique, a Triple-DES (TDES) technique, and a Rivest-Shamir-Adleman (RSA) technique. The encoder 104 may be further operatively coupled to the transmission module 105. The transmission module 105 may be configured to receive the encoded medical imaging data 106 and transmit the encoded medical imaging data 106 to the receiver system 200. The receiver system 200 is further explained in conjunction with FIG. 2.

Referring now to FIG. 2, various modules within the receiver system 200 are illustrated, in accordance with an embodiment of the present disclosure. FIG. 2 is explained in conjunction with FIG. 1. The receiver system 200 receives the encoded medical imaging data 106 from the source system 100, performs various operations on the encoded medical imaging data, and stores the data as decoded medical imaging data 206 which may be used further (for example, for image reconstruction). To perform these operations, the receiver 200 includes a receiver module 201, a decoder 202, a validation module 203, a mapping module 204, and a data store 205.

The receiver system receives the encoded medical imaging data through the receiver module 201. The encoded medical imaging data 106 includes the medical imaging data in a machine-readable format and validated based on a plurality of predefined standards. Encoded medical data generation is already explained in detail in FIG. 1. The plurality of predefined standards is mutually agreed standards for the source system 100 and the receiver system 200. The receiver module 201 is further communicatively coupled to the decoder 202.

The decoder 202 may communicate with the receiver module 201 and receive the encoded medical imaging data 106. Further, the decoder 202 may be configured to decode the encoded medical imaging data 106. For decoding, the decoder 202 may use a decryption technique. For example, the decryption technique may include, but is not limited to, an Advanced Encryption Standard (AES) technique, a Data Encryption Standard (DES) technique, a Triple-DES (TDES) technique, and a Rivest-Shamir-Adleman (RSA) technique. The encoded medical imaging data 106 may be decoded to ensure that neither the encoded medical imaging data 106 is modified nor missed. Further, the decoder 202 may be operatively coupled to the validation module 203.

The validation module 203 may be configured to validate the decoded medical imaging data based on the plurality of predefined standards which is common for the source system 100 and the receiver system 200. The validation may be one of a successful validation or a failed validation. For example, when the decoded medical imaging data is not compatible with the plurality of predefined standards, the validation may fail. Further, an error may be rendered to a user or an administrator along with a reason for failure if the validation of the decoded medical imaging data is failed. In some embodiments the error may be rendered to the user through a user interface. For example, the reason for failure may be rendered to the user via a text or a voice note through a display device. Moreover, when the validation is successful, the validation module 203 may transfer the decoded medical imaging data to the connected mapping module 204.

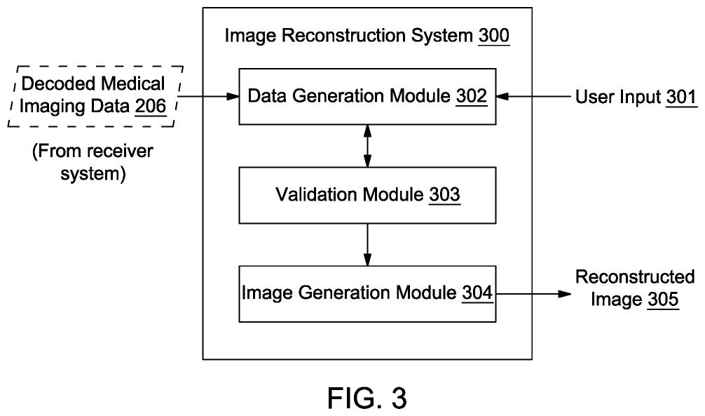

The mapping module 204 may be configured to map the decoded medical imaging data with one or more business objects associated with the receiver system 200. Examples of the one or more business objects may include, but are not limited to, Patient details, protocol parameters, and reconstruction parameters. In some embodiments, for mapping, the mapping module 204 may determine compatibility of the decoded medical imaging data with the receiver system configurations. The mapping module 204 is further operatively coupled to the data store 205. The datastore 205 may store the decoded medical imaging data 206 which may be used further (for example, by the image reconstruction system 300). The image reconstruction system 300 is further explained in conjunction with FIG. 3.

Referring now to FIG. 3, various modules within the image reconstruction system 300 are illustrated, in accordance with an embodiment of the present disclosure. FIG. 3 is explained in conjunction with FIG. 2. The image reconstruction system 300 utilizes the decoded medical imaging data 206 to generate a reconstructed image 305. The reconstructed image 305 may correspond to a reconstructed medical image which may be generated upon requirement using the available decoded imaging data 206 and based on some user inputs. In some embodiments, the reconstructed image 305 may correspond to a Digital Imaging and Communications in Medicine (DICOM) image. To generate the reconstructed image 305, the reconstruction system 300 includes a data generation module 302, a validation moule 303, and an image generation module 304.

In some embodiments, the data generation module 302 may extract the decoded imaging data 206 and receive a user input 301 from the user. Further, the data generation module 302 may be configured to generate reconstruction data based on the stored decoded medical imaging data 206 and the user input 301. For example, the user input 301 may include values corresponding to a size parameter and text corresponding to an orientation parameter of an image to be reconstructed. By way of an example, the user may provide the user input 301 through a Graphical User Interface (GUI) as “generate a medical image with posterior orientation and “35×43 cm size” or “35×43””. Examples of orientation provided as the user input 301 may include, but are not limited to, an axial orientation, coronal orientation, and a sagittal orientation.

In some embodiments a plurality of options corresponding to the size and the orientation parameters may be rendered to the user through the GUI. The user may select one option from the plurality of options. This selected option may be processed to the data generation module 302. Further, based on the selected option and the stored decoded medical imaging data 206, the data generation module 302 may generate the reconstruction data. The data generation module 302 may be further connected to the validation moule 303.

The validation module 303 may be configured to validate the reconstruction data based on a plurality of predefined clinical rules and the set of constant fields. Here, the set of constant fields may include at least one of text data and binary data corresponding to a patient, an imaging system, an institution where the source system is installed, cardiac data, pulmonary data, injector data, a prescribed medicine, and a certified technician.

For example, the constant fields should not be changed in the reconstruction data. By way of an example, consider a scenario where the medical imaging data includes a name ‘A’ of a patient, and in the reconstruction data the name is changed to ‘B’. In that case, the validation may fail, as the constant fields should not be changed. In some embodiments, when the validation is failed the validation module 303 may transmit an error with a reason for failure to the user. In that case the previous step may be performed again. The validation module 303 may be communicatively coupled to the image generation module 302 to transmit the reconstruction data in case the validation is successful.

The image generation module 304 may receive the reconstruction data and based on that generate the reconstructed image 305. In some embodiments, the image generation module 304 may include an Artificial Intelligence (AI) model for generating the reconstructed image 305. Alternatively, in some other embodiments, the image generation module 304 may generate the reconstructed image 305 using some other reconstruction techniques. For example, the reconstruction techniques may include, but are not limited to, an Iterative Model Reconstruction (IMR) technique, an artificial bee colony technique, an iDose technique, a spectral reconstruction technique. It should be noted that the reconstructed image 305 may include information associated with the receiver system 200 as well as information associated with the source system 100.

By way of an example, if a patient resides in a city ‘A’ and getting treatment from a healthcare provider present in the city ‘A’. Further, the patient may decide to relocate to city ‘B’. In that case, the patient needs to change the healthcare provider as the patient may not be able to travel every time to the city ‘A’. Further, for the new healthcare provider, it may not be possible to reuse a previous scan generated by a system associated with the previous healthcare provider when a medical image with new parameters is required. In that case, if the medical imaging data is transferred securely from the previous healthcare provider to the new healthcare provider, it may be easy for the new healthcare provider to reconstruct the medical image with the new parameters.

It should be noted that the systems 100, 200 may be implemented in programmable hardware devices such as programmable gate arrays, programmable array logic, programmable logic devices, or the like. Alternatively, the systems 100, 200 may be implemented in software for execution by various types of processors. An identified engine/module of executable code may, for instance, include one or more physical or logical blocks of computer instructions which may, for instance, be organized as an object, module, procedure, function, or other construct. Nevertheless, the executables of an identified engine/module need not be physically located together but may include disparate instructions stored in different locations which, when joined logically together, comprise the identified engine/module and achieve the stated purpose of the identified engine/module. Indeed, an engine or a module of executable code may be a single instruction, or many instructions, and may even be distributed over several different code segments, among different applications, and across several memory devices.

As will be appreciated by one skilled in the art, a variety of processes may be employed to transmit medical imaging data from a source system 100 to a receiver system 200. For example, the medical imaging data may be transferred from the exemplary system 100 to the exemplary system 200 and further used by the exemplary system 300 for reconstruction, by the process discussed herein. In particular, as will be appreciated by those of ordinary skill in the art, control logic and/or automated routines for performing the techniques and steps described herein may be implemented by the various modules of systems 100, 200 either by hardware, software, or combinations of hardware and software. For example, suitable code may be accessed and executed by the processor associated with the systems 100, 200 to perform some or all of the techniques described herein. Similarly, application specific integrated circuits (ASICs) configured to perform some or all the processes described herein may be included in the processor in the systems 100, 200.

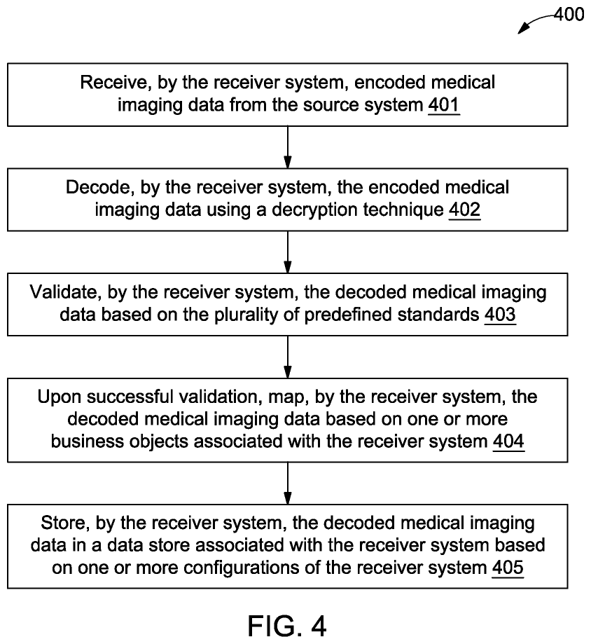

Referring now to FIG. 4, a method for transmitting medical imaging data from a source system to a receiver system is depicted via a flowchart 400, in accordance with an embodiment. Each step of the method may be executed at a receiver system (same as the receiver system 200). FIG. 4 is explained in conjunction with FIGS. 1-3.

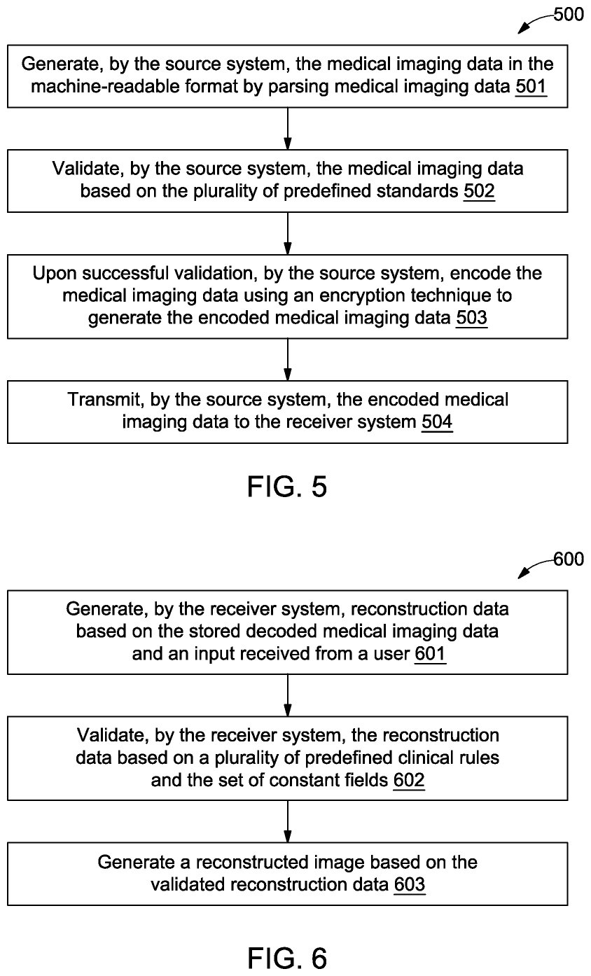

At step 401, encoded medical imaging data (for example, the encoded medical imaging data 106) may be received by the receiver system from the source system (same as the source system 100). It should be noted that the encoded medical imaging data may be ingested by the receiver system through a receiver module (similar to the receiver module 201). Generation of the encoded medical imaging data is explained further in detail conjunction with FIG. 5. The encoded medical imaging data may correspond to the medical imaging data in a machine-readable format and validated based on a plurality of predefined standards. Here, the plurality of predefined standards are common standards for the source system and the receiver system for data transmission. Examples of the plurality of predefined standards may include JavaScript Object Notation (JSON), and Extensible Markup Language (XML).

At step 402, the encoded medical imaging may be decoded. This step may be performed through a decoder (analogous to the decoder 202) of the receiver system. It should be noted that the decoder uses a decryption technique for decoding. Th decryption technique may include, but is not limited to, an Advanced Encryption Standard (AES) technique, a Data Encryption Standard (DES) technique, a Triple-DES (TDES) technique, and a Rivest-Shamir-Adleman (RSA) technique.

At step 403, the decoded medical imaging data may be validated based on the plurality of predefined standards using a validation module (for example, the validation module 203). The plurality of predefined standards is mutually agreed standards for the source system and the receiver system for data transmission. For example, when the decoded medical imaging data is not compliant with the plurality of predefined standards, the validation may be unsuccessful. If the validation of the decoded medical imaging data is unsuccessful, an error may be rendered to a user or an administrator along with a reason for failure. In some embodiments the error may be rendered to the user through a user interface. For example, the reason for failure may be rendered to the user via a text or a voice note through a display device.

For example, when the decoded medical imaging data is compliant with the plurality of predefined standards, the validation may be successful. Further, upon successful validation, at step 404, the decoded medical imaging data may be mapped based on one or more business objects associated with the receiver system. Mapping may be performed using a mapping module (such as the mapping module 204). Thereafter, the decoded medical imaging data may be stored in a data store (for example, the data store 205) associated with the receiver system. It should be noted that according to one or more configurations of the receiver system the decoded medical imaging data may be stored in the data store. In other words, compatibility of the decoded medical imaging data may be determined based on the receiver system configurations. Further, for example, in some embodiments, this stored decoded medical imaging data may be extracted for reconstructing a medical image, which is further explained in conjunction with FIG. 6.

Referring now to FIG. 5, a detailed method for transmitting medical imaging data from a source system is depicted via a flowchart 500, in accordance with an embodiment. Each step of the method may be executed by a source system (same as the source system 100). FIG. 5 is explained in conjunction with FIGS. 1-4.

At step 501, the medical imaging data (for example, the medical imaging data 101) may be generated in a machine-readable format. In some embodiments, to generate the medical imaging data in the machine-readable format, the medical imaging data may be parsed using a parser (for example, the parser 102). It should be noted that the medical imaging data may include a set of variable fields and a set of constant fields. The set of variable fields may include at least one of text data and binary data corresponding to an examination parameter, a scan preference, an image acquisition parameter (for example, orientation), a bolus tracking detail, collimation data, a scan geometry, scan coordinates, one or more examination rules and constraints, and additional examination data. And the set of constant fields may include at least one of text data and binary data corresponding to a patient, an imaging system, an institution where the source system is installed, cardiac data, pulmonary data, injector data, a prescribed medicine, and a certified technician.

At step 502, the medical imaging data may be validated based on the plurality of predefined standards. This step may be performed using a validation module (such as the validation module 103). The plurality of predefined standards is mutually agreed standards for the source system and the receiver system for data transmission. For example, when the format of the medical imaging data is not compliant with the plurality of predefined standards, the validation may be unsuccessful. If the validation of the medical imaging data is unsuccessful, an error may be rendered to the user or the administrator along with a reason for failure. In some embodiments, the error may be rendered to the user through a user interface. For example, the reason for failure may be rendered to the user via a text or a voice note through a display device.

For example, when the medical imaging data is compliant with the plurality of predefined standards, the validation may be successful. Further, upon successful validation, at step 503, the medical imaging data may be encoded through an encoder (such as the encoder 104). The encoder uses an encryption technique to generate the encoded medical imaging data. For example, the encryption technique may include, but is not limited to, an Advanced Encryption Standard (AES) technique, a Data Encryption Standard (DES) technique, a Triple-DES (TDES) technique, and a Rivest-Shamir-Adleman (RSA) technique. Thereafter, at step 504, the encoded medical imaging data may be transmitted to the receiver system.

Referring now to FIG. 6, a method of reconstructing a medical image is depicted via a flowchart 600, in accordance with an embodiment. Each step of the method may be executed by an image reconstruction system (same as the image reconstruction system 300). FIG. 6 is explained in conjunction with FIGS. 1-5.

At step 601, reconstruction data may be generated based on the stored decoded medical imaging data and an input (same as the user input 301) received from a user. To generate the reconstruction data, in some embodiments, decoded medical imaging data may be extracted from the data store. A data generation module (such as the data generation module 302) may be used to generate the reconstruction data. It should be noted that the input may include values corresponding to at least one of a size parameter and an orientation parameter of an image to be constructed.

At step 602, the reconstruction data may be validated based on a plurality of predefined clinical rules and the set of constant fields. A validation module (such as the validation module 303) may be employed to perform this step. The set of constant fields may include at least one of text data and binary data corresponding to a patient, an imaging system, an institution where the source system is installed, cardiac data, pulmonary data, injector data, a prescribed medicine, and a certified technician. For example, the constant fields should not be changed in the reconstruction data. By way of an example, consider a scenario where the medical imaging data includes a name ‘A’ of a patient, and in the reconstruction data the name is changed to ‘B’. In that case, the validation may fail, as the constant fields should not be changed. In some embodiments, when the validation is failed an error with a reason for failure may be rendered to the user. In that case the previous step may be performed again.

Further, in case the validation is successful, at step 603, a reconstructed image (such as the reconstructed image 305) may be generated based on the validated reconstruction data using an image generation module (same as the image generation module 304). In some embodiments, the image generation module may include an Artificial Intelligence (AI) model for generating the reconstructed image. Alternatively, in some other embodiments, the reconstructed image may be generated using other reconstruction techniques. For example, the reconstruction techniques may include, but are not limited to, an Iterative Model Reconstruction (IMR) technique, an artificial bee colony technique, an iDose technique, a spectral reconstruction technique. It should be noted that the reconstructed image may include information associated with the source system and the receiver system.

By way of an example, a patient goes to a government hospital for treatment. But now the patient has decided to switch the hospital to a private hospital. Now, the patient has noisy medical images scanned at the government hospital. In that case, a doctor from the private hospital may not be able to understand disease using those noisy medical images. So, the doctor may recommend the patient to go for another scan. Contrast material used or scanning may affect kidney health of the patient. To avoid the use of contrast material again and again, the disclosure provides the processes discussed above in FIGS. 4-6 of transferring the medical imaging data securely which may be used in these types of scenarios for image reconstructions.

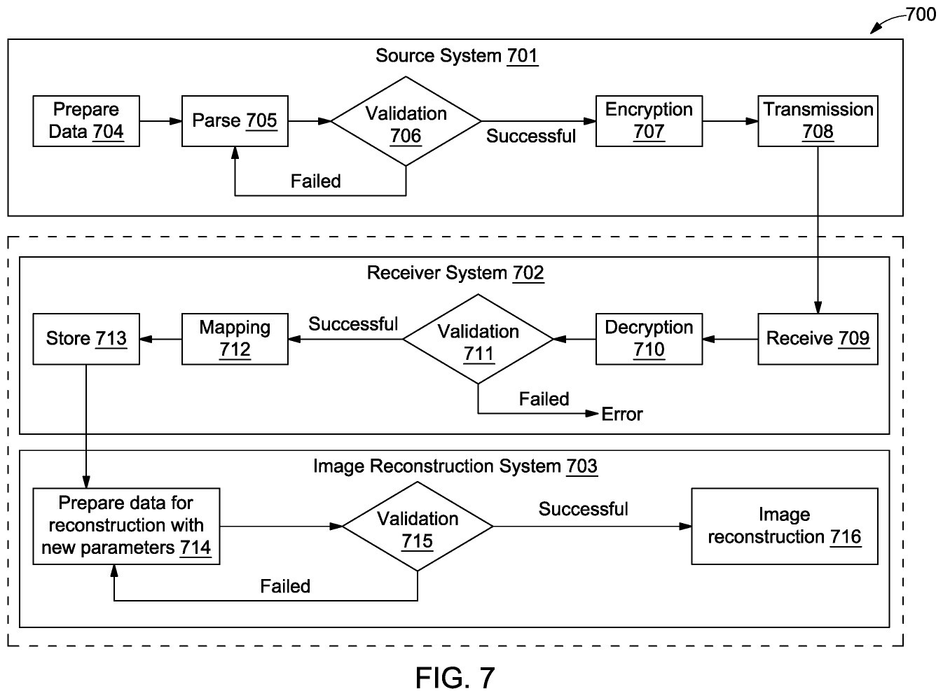

Referring now to FIG. 7, a control logic 700 for transmitting medical imaging data from a source system 701 to a receiver system 702 is illustrated, in accordance with an embodiment. Each step of the control logic 700 may be executed by the source system 701, the receiver system 702, and an image reconstruction 703 (same as the source system 100, the receiver system 200, and the image reconstruction system 300). FIG. 7 is explained in conjunction with FIGS. 1-6.

At step 704, medical imaging data may be prepared. The medical imaging data may include a set of constant fields and a set of variable fields. For example, the set of constant fields may include data corresponding to a patient, an imaging system, an institution where the source system is installed, cardiac data, pulmonary data, injector data, a prescribed medicine, and a certified technician. The set of variable fields may include data corresponding to an examination parameter, a scan preference, an image acquisition parameter, a bolus tracking detail, collimation data, a scan geometry, scan coordinates, one or more examination rules and constraints, and additional examination data. The medical imaging data may be at least one of text data and binary data.

At step 705, the medical imaging data may be parsed to obtain the medical imaging data in a machine-readable format. Thereafter, at step 706, the medical imaging data may be validated based on a plurality of predefined standards to ensure that the medical imaging data is in the machine readable format. The plurality of predefined standards may be common standards for the source system 701 and the receiver system 702. For example, the format of the medical imaging data should be compliant with both the source system 701 and the receiver system 702.

At step 707, the validated medical imaging data may be encrypted using an encryption technique. Examples of the encryption technique may include, but are not limited to, the AES technique, the DES technique, the T-DES technique, and the RSA technique. Hence, encoded medical imaging data may be obtained because of encryption. The encoded medical imaging data may be transmitted to the receiver system 702, at step 708.

At step 709, the encoded medical imaging data may be received. At step 710, the encoded medica imaging data may be decrypted through a decryption technique. Thus, decoded medical imaging data may be obtained in response to decryption. After that, at step 711, the decoded medical imaging data may be validated based on the plurality of predefined standards to ensure that the neither the encoded medical imaging data is modified or lost during data transmission. In case the validation is unsuccessful, an error may be rendered. For example, when a change is identified in received data and transmitted data, the validation may be unsuccessful.

At step 712, upon successful validation, the decoded medical imaging data may be mapped based on business objects associated with the receiver system 702 ensuring compatibility of the decoded medical imaging data with the receiver system configurations. At step 714, the decoded medical imaging data may be stored in a data store associated with the receiver system 702 based on one or more configurations of the receiver system.

At step 714, data may be prepared with new parameters for image reconstruction. For example, the decoded medical imaging data may be extracted, and a user input may be received to prepare the data with new parameters. The data with new parameters may also be referred to as reconstruction data. The set of variable fields in the decoded medical imaging data may be changed based on the user input. For example, the input may include values corresponding to at least one of a size parameter and an orientation parameter of an image to be reconstructed.

Thereafter, at step 715, the reconstruction data may be validated based on a plurality of predefined clinical rules and the set of constant fields. For example, when data corresponding to any of the constant fields is changed, the validation may be unsuccessful or failed. Further, if the validation is successful, an image reconstruction process may be executed to generate a reconstructed image based on the validated reconstruction data through an AI or any other image reconstruction technique. The reconstructed image may correspond to a DICOM image. The reconstructed image may include information associated with the source system 701 and the receiver system 702. In some embodiments, the image reconstruction system 703 may be a part of the receiver system 702. Alternatively, in some other embodiments, the image reconstruction system 703 may be communicatively connected to the receiver system 702.

For example, sometimes one healthcare provider may have a system that lacks in constructing images with high quality. In that case, the medical imaging data may be transferred to another system that may be associated with another health care provider and images may be reconstructed with higher parameters.

The present disclosure is about exchanging raw medical imaging data rather than transmitting the DICOM images. It should be noted that there may be a difference in DICOM images scanned by two different scanners (scanned at the same time for the same patient but due to different system configurations). Therefore, rather than transferring the DICOM images transferring the medical imaging data is more efficient process. The present disclosure provides a platform through which image reconstruction is possible even if the source system and the receiver system have different types of scanners. The platform can be used for various medical imaging techniques such as Computed Tomography (CT) scans, Magnetic Resonance Imaging (MRI), Positron-emission Tomography (PET), and the like.

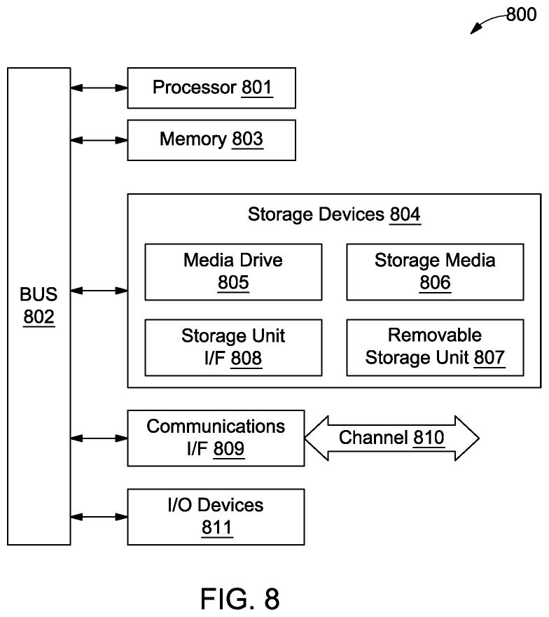

The disclosed methods and systems may be implemented on a conventional or a general-purpose computer system, such as a personal computer (PC) or server computer. Referring now to FIG. 8, an exemplary computing system 800 that may be employed to implement processing functionality for various embodiments (e.g., as a SIMD device, client device, server device, one or more processors, or the like) is illustrated. Those skilled in the relevant art will also recognize how to implement the invention using other computer systems or architectures. The computing system 800 may represent, for example, a user device such as a desktop, a laptop, a mobile phone, personal entertainment device, DVR, and so on, or any other type of special or general-purpose computing device as may be desirable or appropriate for a given application or environment. The computing system 800 may include one or more processors, such as a processor 801 that may be implemented using a general or special purpose processing engine such as, for example, a microprocessor, microcontroller or other control logic. In this example, the processor 801 is connected to a bus 802 or other communication medium. In some embodiments, the processor 801 may be an AI processor, which may be implemented as a Tensor Processing Unit (TPU), or a graphical processor unit, or a custom programmable solution Field-Programmable Gate Array (FPGA).

The computing system 800 may also include a memory 803 (main memory), for example, Random Access Memory (RAM) or other dynamic memory, for storing information and instructions to be executed by the processor 801. The memory 803 also may be used for storing temporary variables or other intermediate information during execution of instructions to be executed by the processor 801. The computing system 800 may likewise include a read only memory (“ROM”) or other static storage device coupled to bus 802 for storing static information and instructions for the processor 802.

The computing system 800 may also include a storage device 804, which may include, for example, a media drives 805 and a removable storage interface. The media drive 805 may include a drive or other mechanism to support fixed or removable storage media, such as a hard disk drive, a floppy disk drive, a magnetic tape drive, an SD card port, a USB port, a micro USB, an optical disk drive, a CD or DVD drive (R or RW), or other removable or fixed media drive. A storage media 806 may include, for example, a hard disk, magnetic tape, flash drive, or other fixed or removable medium that is read by and written to by the media drive 805. As these examples illustrate, the storage media 806 may include a computer-readable storage medium having stored there in particular computer software or data.

In alternative embodiments, the storage devices 804 may include other similar instrumentalities for allowing computer programs or other instructions or data to be loaded into the computing system 800. Such instrumentalities may include, for example, a removable storage unit 807 and a storage unit interface 808, such as a program cartridge and cartridge interface, a removable memory (for example, a flash memory or other removable memory module) and memory slot, and other removable storage units and interfaces that allow software and data to be transferred from the removable storage unit 807 to the computing system 800.

The computing system 800 may also include a communications interface 818. The communications interface 809 may be used to allow software and data to be transferred between the computing system 800 and external devices. Examples of the communications interface 809 may include a network interface (such as an Ethernet or other NIC card), a communications port (such as for example, a USB port, a micro USB port), Near field Communication (NFC), etc. Software and data transferred via the communications interface 809 are in the form of signals which may be electronic, electromagnetic, optical, or other signals capable of being received by the communications interface 809. These signals are provided to the communications interface 809 via a channel 810. The channel 810 may carry signals and may be implemented using a wireless medium, wire or cable, fiber optics, or other communications medium. Some examples of the channel 810 may include a phone line, a cellular phone link, an RF link, a Bluetooth link, a network interface, a local or wide area network, and other communications channels.

The computing system 800 may further include Input/Output (I/O) devices 811. Examples may include, but are not limited to a display, keypad, microphone, audio speakers, vibrating motor, LED lights, etc. The I/O devices 811 may receive input from a user and also display an output of the computation performed by the processor 801. In this document, the terms “computer program product” and “computer-readable medium” may be used generally to refer to media such as, for example, the memory 803, the storage devices 804, the removable storage unit 807, or signal(s) on the channel 810. These and other forms of computer-readable media may be involved in providing one or more sequences of one or more instructions to the processor 801 for execution. Such instructions, generally referred to as “computer program code” (which may be grouped in the form of computer programs or other groupings), when executed, enable the computing system 800 to perform features or functions of embodiments of the present invention.

In an embodiment where the elements are implemented using software, the software may be stored in a computer-readable medium and loaded into the computing system 800 using, for example, the removable storage unit 807, the media drive 805 or the communications interface 809. The control logic (in this example, software instructions or computer program code), when executed by the processor 801, causes the processor 801 to perform the functions of the invention as described herein.

It is intended that the disclosure and examples be considered as exemplary only, with a true scope and spirit of disclosed embodiments being indicated by the following claims

What is claimed is:

1. A method of transmitting medical imaging data from a source system to a receiver system, the method comprising:
receiving, by the receiver system, encoded medical imaging data from the source system, wherein the encoded medical imaging data comprises the medical imaging data in a machine-readable format and validated based on a plurality of predefined standards;
decoding, by the receiver system, the encoded medical imaging data using a decryption technique;
validating, by the receiver system, the decoded medical imaging data based on the plurality of predefined standards;
upon successful validation, mapping, by the receiver system, the decoded medical imaging data based on one or more business objects associated with the receiver system; and
storing, by the receiver system, the decoded medical imaging data in a data store associated with the receiver system based on one or more configurations of the receiver system.

2. The method of claim 1, comprising:
generating, by the source system, the medical imaging data in the machine-readable format by parsing medical imaging data, wherein the medical imaging data comprises a set of variable fields and a set of constant fields;
validating, by the source system, the medical imaging data based on the plurality of predefined standards;
upon successful validation, by the source system, encoding the medical imaging data using an encryption technique to generate the encoded medical imaging data; and
transmitting, by the source system, the encoded medical imaging data to the receiver system.

3. The method of claim 2, wherein the set of constant fields comprises at least one of text data and binary data corresponding to a patient, an imaging system, an institution where the source system is installed, cardiac data, pulmonary data, injector data, a prescribed medicine, and a certified technician.

4. The method of claim 2, wherein the set of variable fields comprises at least one of text data and binary data corresponding to an examination parameter, a scan preference, an image acquisition parameter, a bolus tracking detail, collimation data, a scan geometry, scan coordinates, one or more examination rules and constraints, and additional examination data.

5. The method of claim 2, comprising:
generating, by the receiver system, reconstruction data based on the stored decoded medical imaging data and an input received from a user, wherein the input comprises values corresponding to at least one of a size parameter and an orientation parameter of an image; and
validating, by the receiver system, the reconstruction data based on a plurality of predefined clinical rules and the set of constant fields.

6. The method of claim 5, comprising generating a reconstructed image based on the validated reconstruction data, wherein the reconstructed image comprises information associated with the source system and the receiver system.

7. The method of claim 5, further comprising rendering an error with a reason of failure to the user, for each failed validation corresponding to the medical imaging data, the decoded medical imaging data, and the reconstruction data.

8. The method of claim 1, wherein storing the decoded medical imaging data comprises determining compatibility of the decoded medical imaging data with the receiver system configurations.

9. A system for transmitting medical imaging data from a source system to a receiver system, the system comprising:
a processor; and
a memory communicatively coupled to the processor, wherein the memory stores processor-executable instructions, which, on execution, cause the processor to:
receive encoded medical imaging data from the source system,
wherein the encoded medical imaging data comprises the medical imaging data in a machine-readable format and validated based on a plurality of predefined standards;
decode the encoded medical imaging data using a decryption technique;
validating, by the receiver system, the decoded medical imaging data based on the plurality of predefined standards;
upon successful validation, map the decoded medical imaging data based on one or more business objects associated with the receiver system; and
store the decoded medical imaging data in a data store associated with the receiver system based on one or more configurations of the receiver system.

10. The system of claim 9, wherein the processor-executable instructions further cause the processor to:
generate the medical imaging data in the machine-readable format by parsing medical imaging data, wherein the medical imaging data comprises a set of variable fields and a set of constant fields, wherein the set of constant fields comprises at least one of text data and binary data corresponding to a patient, an imaging system, an institution where the source system is installed, cardiac data, pulmonary data, injector data, a prescribed medicine, and a certified technician, and wherein the set of variable fields comprises at least one of text data and binary data corresponding to an examination parameter, a scan preference, an image acquisition parameter, a bolus tracking detail, collimation data, a scan geometry, scan coordinates, one or more examination rules and constraints, and additional examination data;
validate the medical imaging data based on the plurality of predefined standards;
upon successful validation, encode the medical imaging data using an encryption technique to generate the encoded medical imaging data; and
transmit the encoded medical imaging data to the receiver system.

11. The system of claim 10, wherein the processor-executable instructions further cause the processor to:
generate reconstruction data based on the stored decoded medical imaging data and an input received from a user, wherein the input comprises values corresponding to at least one of a size parameter and an orientation parameter of an image;
validate the reconstruction data based on a plurality of predefined clinical rules and the set of constant fields; and
generate a reconstructed image based on the validated reconstruction data, wherein the reconstructed image comprises information associated with the source system and the receiver system.
